(19) United States
(12) Patent Application Publication
(10) Pub. No.: US 2008/0306571 A1
(43) Pub. Date: Dec. 11, 2008

(54) Devices For Treatment of Central Nervous System Injuries

(76) Inventors: Richard B. Borgens, Delphi, IN (US); Pedro Irazoqui, Lafayette, IN (US); John M. Cirillo, Oxford, IN (US)

Correspondence Address: Thomas A Walsh;Ice Miller, One American Square Suite3100, Indianapolis, IN, 46282-0200, US

(21) Appl. No.: 11/919,784

(22) PCT Filed: Mar. 16, 2006
(86) PCT No.: PCT/US2006/009383

§ 371 Date: Nov. 1, 2007

Related U.S. Application Data
(60) Provisional application No. 60/662,495, filed on Mar. 16, 2005.
(60) Provisional application No. 60/719,915, filed on Sep. 23, 2005.
(60) Provisional application No. 60/719,911, filed on Sep. 23, 2005.
(60) Provisional application No. 60/719,818, filed on Sep. 23, 2005.

(51) Int. Cl. A61N 1/00

(52) U.S. Cl. 607/50

(57) ABSTRACT

A device for stimulating axon growth of the nerve cells in the spinal cord of mammals includes a device having a DC stimulus generator (356, 358, 360, 362, 364, 366, 388, 390, 368, 370, 372, 374, 376, 378, 392, 394, 606, 608) having first and second oppositely polarized output terminals (341, 343, 345, 385, 648; 347, 349, 351, 387 646), first and second electrodes (340, 342, 344, 384, 612; 346, 348, 350, 386, 610) electrically coupled to the first and second terminals respectively, and a polarity reversing circuit (382, 380; 602, 604) electrically connected to the constant current DC stimulus generator and configured to reverse the polarity of the DC stimulus each time a predetermined period of time elapses. A method for stimulating axon growth of the nerve cells in the spinal cord of mammals includes providing such a device and implanting the provides device in a mammal.

### PRIORITY CLAIM

This application claims priority to U.S. Provisional Application Ser. No. 60/662,495 entitled DEVICES FOR TREATMENT OF CENTRAL NERVOUS SYSTEM INJURIES, by Richard B. Borgens and John M Cirillo filed Mar. 16, 2005; U.S. Provisional Application Ser. No. 60/719,915 entitled RECHARGEABLE SYSTEM FOR TREATMENT OF NEURAL INJURIES, by Richard B. Borgens, Pedro Irazoqui and John M Cirillo filed Sep. 23, 2005; U.S. Provisional Application Ser. No. 60/719,911 entitled ENCASED SYSTEM FOR TREATMENT OF NEURAL INJURIES, by Richard B. Borgens, Pedro Irazoqui and John M Cirillo filed Sep. 23, 2005; and, U.S. Provisional Application Ser. No. 60/719,818 entitled SYSTEM FOR TREATMENT OF MOTOR NEURON INJURIES, by Richard B. Borgens, Pedro Irazoqui and John M Cirillo, filed Sep. 23, 2005.

### BACKGROUND

This disclosure relates generally to devices and methods for stimulating nerve cell regeneration and more particularly to devices and methods for stimulating nerve cell regeneration in the central nervous system of mammals through the application of oscillating DC electrical fields.

Injury to the spinal cord or central nervous system can be one of the most devastating and disabling injuries possible. Depending upon the severity of the injury, paralysis of varying degrees can result. Paraplegia and quadriplegia often result from severe injury to the spinal cord. The resulting effect on the sufferer, be it man or animal, is severe. The sufferer can be reduced to a state of near immobility or worse. For humans, the mental trauma induced by such severe physical disability can be even more devastating than the physical disability itself.

When the spinal cord of a mammal is injured, connections between nerves in the spinal cord are broken. The injured portion of the spinal cord is termed a “lesion.” Such lesions block the flow of nerve impulses for the nerve tracts affected by the lesion with resulting impairment to both sensory and motor function.

To restore the lost sensory and motor functions, the affected motor and sensory axons of the injured nerves must regenerate, that is, grow back. Unfortunately, any spontaneous regeneration of injured nerves in the central nervous system of mammals has been found to occur, if at all, only within a very short period immediately after the injury occurs. After this short period expires, such nerves have not been found to regenerate further spontaneously.

Studies have shown, however, that the application of a DC electrical field across a lesion and the damaged nerve ending adjacent the lesion in the spinal cord of mammals, can promote axon growth, and the axons will grow back around the lesion. Since the spinal cord is rarely severed completely when injured, the axons need not actually grow across the lesion but can circumnavigate the lesion through remaining spinal cord parenchyma.

Although axon growth can be promoted by the application of a steady DC electrical field, only those axons facing the cathode (negative pole) are stimulated to grow. Axons facing the anode (positive pole) not only are not stimulated to grow, but actually reabsorb into the bodies of the nerve cells (“die back),” after a period of time. In order to “repair” an injured spinal cord, regeneration of both the ascending and descending nerve tracks must be promoted. Thus, axons growth in both directions, i.e., rostrally and caudally, must be stimulated to “repair” an injured spinal cord.

For optimal results in a human patient, a uniform electrical field of a desired strength is imposed over about 10 cm to 20 cm of damaged spinal cord for a beneficial clinical outcome. Ideally, this uniform field is imposed across the entire cross section of the spinal cord over this longitudinal extent, because of the general segregation of descending (motor) tracts to the ventral (anterior) cord, and the segregation of important (largely sensory) tracts to the posterior (dorsal) spinal cord. In paraplegic canines, this electrical field has been directly measured (Richard B. Borgens, James P. Toombs, Andrew R. Blight, Michael E. McGinnis, Michael S. Bauer, William R. Widmer, and James R. Cook Jr., Effects of Applied Electric Fields on Clinical Cases of Complete Paraplegia in Dogs, J. Restorative Neurology and Neurosci., 1993, pp. 5:305-322). In man however, the cross sectional area of the spinal cord is approximately two to four times that of the small to medium sized dogs treated in clinical trials, and actual invasive measurement of the imposed electrical fields is not feasible on human patients.

Based on the responses of human paraplegics and quadriplegics to prior art therapies involving the application of an oscillating DC electrical field across a lesion in the spinal cord using three pairs of electrodes, it appears that the dorsal (posterior) location of three pairs of electrodes did not produce a uniform field over the entire unit area of the patient's spinal cord. This was revealed by the domination of sensory recovery in these patients (greater than thirty fold over historical controls) compared to motor recovery (approximately twofold greater than historical controls) using the ASIA scoring system. Thus, this result indicates that when the prior treatment method is utilized the voltage gradient was highest nearest to the actual location of electrode placement. In the prior method of treatment two pairs of electrodes were placed on either side (two tethered to the right and left lateral facets) and a third pair was sutured to the paravertebral muscle and fascia of the dorsal (posterior) facet rostrally and caudally of the spinal cord lesion (Shapiro, et al., Oscillating Field Stimulation for Complete Spinal Cord Injury in Humans: a Phase 1 Trial, Journal of Neurosurg. Spine 2, 2005, pp. 3-10).

It would be desirable to provide a device to generate a stronger DC electrical field across the spinal cord lesion and the areas adjacent thereto (over the entire cross-sectional area of the spinal cord and the intact areas bordering the lesion rostrally and caudally) of a human in order to facilitate the creation of a uniform electrical field over the entire affected area. It would be further desirable to provide a method for implanting electrodes that facilitates the creation of a uniform electrical field over the affected area of the injured spinal cord.

Existing devices to generate a DC electrical field across a lesion and areas adjacent thereto in the spinal cord of mammals are implanted into the patient, and powered by a battery. These batteries are sealed and are not readily rechargeable. Therefore, when a patient could benefit from longer terms of treatment, either a larger battery must be used, or the device must be removed from the patient and replaced via a surgery. It would be desirable to provide a device to generate the DC electrical field across the spinal cord lesion and the areas adjacent thereto that has a smaller battery, a battery with a longer useful life, or both.

The devices of the existing technology are shielded from the biology with Teflon. Over time, Teflon may allow seepage of bodily fluids into the device, which would in turn lead to chemical compounds from the device being absorbed by the surrounding tissue. It would be desirable to provide a case for a device that acts as a persistent barrier between the circuitry of the device and the surrounding tissue.

### SUMMARY

According to one aspect of the disclosure, an apparatus for stimulating axon growth of the nerve cells in the spinal cord of mammals comprises a constant current DC stimulus generator, first and second groups of electrodes, a beacon signal generator and a polarity reversing circuit. The constant current DC stimulus generator has first and second groups of oppositely polarized output terminals. The one of the first or second groups of output terminals comprises a cathode and the other one of the first or second groups of output terminals comprises an anode of the generator. The first and second groups of electrodes are electrically coupled respectively to the first and second groups of output terminals. The beacon signal generator is electrically coupled to the DC stimulus generator. The polarity reversing circuit is electrically coupled to the constant current DC stimulus generator and is configured to reverse the polarity of the DC stimulus each time a predetermined period of time elapses. Each time the polarity of the DC stimulus is reversed the output terminal which comprised the cathode before the polarity reversal comprises the anode after the reversal and the output terminal which comprised the anode before the polarity reversal comprises the cathode after the polarity reversal.

According to another aspect of the disclosure, an apparatus for stimulating axon growth of the nerve cells in the spinal cord of mammals comprises a stimulus generator, first and second electrodes and a polarity reversing circuit. The stimulus generator is capable of generating a chopped DC current and has first and second oppositely polarized output terminals. The one of the first or second output terminals comprises a cathode and the other one of the first or second output terminals comprises an anode of the generator. The first and second electrodes are electrically coupled respectively to the first and second output terminals. The polarity reversing circuit is electrically coupled to the stimulus generator and is configured to reverse the polarity of the stimulus each time a predetermined period of time elapses. Each time the polarity of the DC stimulus is reversed the output terminal which comprised the cathode before the polarity reversal comprises the anode after the reversal and the output terminal which comprised the anode before the polarity reversal comprises the cathode after the polarity reversal.

According to yet another aspect of the disclosure, a method for stimulating axon growth of the nerve cells in the spinal cord of mammals comprises providing a device and implanting the device in a mammal. The provided device comprises a constant current DC stimulus generator, first and second groups of electrodes, a beacon signal generator and a polarity reversing circuit. The constant current DC stimulus generator has first and second groups of oppositely polarized output terminals. The one of the first or second groups of output terminals comprises a cathode and the other one of the first or second groups of output terminals comprises an anode of the generator. The first and second groups of electrodes are electrically coupled respectively to the first and second groups of output terminals. The beacon signal generator is electrically coupled to the DC stimulus generator. The polarity reversing circuit is electrically coupled to the constant current DC stimulus generator and is configured to reverse the polarity of the DC stimulus each time a predetermined period of time elapses. Each time the polarity of the DC stimulus is reversed the output terminal which comprised the cathode before the polarity reversal comprises the anode after the reversal and the output terminal which comprised the anode before the polarity reversal comprises the cathode after the polarity reversal.

According to yet another aspect of the disclosure, a method for stimulating axon growth of the nerve cells in the spinal cord of mammals comprises providing a device and implanting the device in a mammal. The provided device comprises a stimulus generator, first and second groups of electrodes and a polarity reversing circuit. The stimulus generator is capable of generating a chopped DC current and has first and second groups of oppositely polarized output terminals. The one of the first or second groups of output terminals comprises a cathode and the other one of the first or second groups of output terminals comprises an anode of the generator. The first and second groups of electrodes are electrically coupled respectively to the first and second groups of output terminals. The polarity reversing circuit is electrically coupled to the stimulus generator and is configured to reverse the polarity of the stimulus each time a predetermined period of time elapses. Each time the polarity of the stimulus is reversed the output terminal which comprised the cathode before the polarity reversal comprises the anode after the reversal and the output terminal which comprised the anode before the polarity reversal comprises the cathode after the polarity reversal.

According to another aspect of the disclosure, an apparatus for stimulating axon growth of the nerve cells in the spinal cord of mammals comprises a constant current DC stimulus generator, first and second groups of electrodes, a rechargeable charge storage device, and a polarity reversing circuit. The constant current DC stimulus generator has first and second groups of oppositely polarized output terminals. One of the first or second groups of output terminals comprises a cathode and the other one of the first or second groups of output terminals comprises an anode of the generator. The first and second groups of electrodes are electrically coupled respectively to the first and second groups of output terminals. The rechargeable charge storage device is electrically coupled to the constant current DC stimulus generator. The polarity reversing circuit is electrically coupled to the constant current DC stimulus generator and is configured to reverse the polarity of the DC stimulus each time a predetermined period of time elapses. Each time the polarity of the DC stimulus is reversed the output terminal which comprised the cathode before the polarity reversal comprises the anode after the reversal and the output terminal which comprised the anode before the polarity reversal comprises the cathode after the polarity reversal.

According to another aspect of the disclosure, an apparatus for stimulating axon growth of the nerve cells in the spinal cord of mammals comprises a constant current DC stimulus generator, first and second groups of electrodes, a charge storage device, a case and a polarity reversing circuit. The constant current DC stimulus generator has first and second groups of oppositely polarized output terminals. One of the first or second groups of output terminals comprises a cathode and the other one of the first or second groups of output terminals comprises an anode of the generator. The first and second groups of electrodes are electrically coupled respectively to the first and second groups of output terminals. The charge storage device is electrically coupled to the constant current DC stimulus generator. The case has a top portion and a bottom portion. The constant current DC stimulus generator and the charge storage device are positioned between the top portion and bottom portion. The polarity reversing circuit is electrically coupled to the constant current DC stimulus generator and is configured to reverse the polarity of the DC stimulus each time a predetermined period of time elapses. Each time the polarity of the DC stimulus is reversed the output terminal which comprised the cathode before the polarity reversal comprises the anode after the reversal and the output terminal which comprised the anode before the to polarity reversal comprises the cathode after the polarity reversal.

According to yet another aspect of the disclosure, an apparatus implanted in a mammalian body having a spine and a lesion in the spinal cord for stimulating axon growth of the nerve cells in the spinal cord of mammals comprises a constant current DC stimulus generator, First and second groups of electrodes, and a polarity revering circuit. The constant current DC stimulus generator has first and second groups of oppositely polarized output terminals wherein one of the first and second groups of output terminals comprises a cathode and the other of the first and second groups of output terminals comprises an anode of the generator. The first and second groups of electrodes are electrically coupled respectively to the first and second groups of output terminals. Each of said first and second groups of electrodes having a first electrode corresponding to a first electrode of the other of the first and second groups, a second electrode corresponding to a second electrode of the other of the first and second groups, a third electrode corresponding to a third electrode of the other of the first and second groups, and a fourth electrode corresponding to a fourth electrode of the other of the first and second groups. The polarity reversing circuit is electrically coupled to the constant current DC stimulus generator and is configured to reverse the polarity of the DC stimulus each time a predetermined period of time elapses. Each time the polarity of the DC stimulus is reversed the output terminal which comprised the cathode before the polarity reversal comprises the anode after the reversal and the output terminal which comprised the anode before the polarity reversal comprises the cathode after the polarity reversal. The first electrodes of the first and second group of electrodes are positioned on the right lateral facet of the spine of the mammalian body with the electrode of the first group of electrodes positioned rostrally from the lesion and the electrode of the second group of electrodes positioned caudally from the lesion. The second electrodes of the first and second group of electrodes are positioned on the left lateral facet of the spine of the mammalian body with the electrode of the first group of electrodes positioned rostrally from the lesion and the electrode of the second group of electrodes positioned caudally from the lesion, the third electrodes of the first and second group of electrodes are positioned on the paravertebral muscle and fascia of the dorsal (posterior) facet of the mammalian body with the electrode of the first group of electrodes positioned rostrally from the lesion and the electrode of the second group of electrodes positioned caudally from the lesion, and the fourth electrodes of the first and second group of electrodes are positioned adjacent to paravertebral musculature at the extreme mediolateral/ventral (anterior) vertebral column of the mammalian body with the electrode of the first group of electrodes positioned rostrally from the lesion and the electrode of the second group of electrodes positioned caudally from the lesion.

Additional features and advantages will become apparent to those skilled in the art upon consideration of the following detailed description of a preferred embodiment exemplifying the best mode of carrying out the invention as presently perceived.

### BRIEF DESCRIPTION OF THE DRAWINGS

The features and advantages of the disclosed devices, and the methods of obtaining them, will be more apparent and better understood by reference to the following descriptions of embodiments of the devices, taken in conjunction with the accompanying drawings, wherein:

FIG. 1 shows a graph that portrays the effect of an applied steady DC field over time on the growth of cathodal and anodal facing axons;

FIG. 2 shows a graph that portrays the effect of an applied oscillating field over time on the growth of cathodal and anodal facing axons;

FIG. 3 shows a schematic of a first embodiment of a circuit for generating an oscillating electrical field for stimulating nerve regeneration;

FIG. 4 shows a schematic of a beacon circuit for use in conjunction with a circuit for generating an oscillating electrical field for stimulating nerve regeneration;

FIG. 5 shows a schematic of a receiver circuit for use in conjunction with the beacon circuitry of FIG. 4;

FIG. 6 shows a schematic of a second embodiment of a circuit for generating an oscillating electrical field for stimulating nerve regeneration;

FIG. 7A shows a first portion of a schematic of an embodiment of a circuit having eight electrodes for generating an oscillating electrical field for stimulating nerve regeneration;

FIG. 7B shows a second portion of a schematic of the embodiment of a circuit having eight electrodes for generating an oscillating electrical field for stimulating nerve regeneration;

FIG. 8 shows a schematic of a rechargeable circuit for generating an oscillating electrical field for stimulating nerve regeneration;

FIG. 9 shows a detailed portion of the schematic of the rechargeable circuit for generating an oscillating electrical field for stimulating nerve regeneration of FIG. 8;

FIG. 10 shows a block diagram of the rechargeable circuit for generating an oscillating electrical field for stimulating nerve regeneration of FIG. 8;

FIG. 11 shows a perspective view of a case for use with the circuit of either FIG. 3, 6, 7, 8, 9 or 10; and

FIG. 12 shows a graph that portrays the effect of an applied pulse wave modulated oscillating field over time on the growth of cathodal and anodal facing axons.

### DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present invention includes any alterations and modifications to the illustrated embodiments and includes further applications of the principles of the disclosure as would normally occur to one skilled in the art to which this invention pertains.

The application of an oscillating DC electrical field across a lesion and the area adjacent the lesion in the spinal cord of a mammal can stimulate axon growth in both directions, i.e., caudally and rostrally. That is, growth of caudally facing axons will be promoted as will growth of rostrally facing axons. The DC electrical field is a constant current stimulus which is first applied in one direction for a predetermined period of time and then applied in the opposite direction for the predetermined period of time. The polarity of the constant current DC stimulus is reversed after each predetermined period of time.

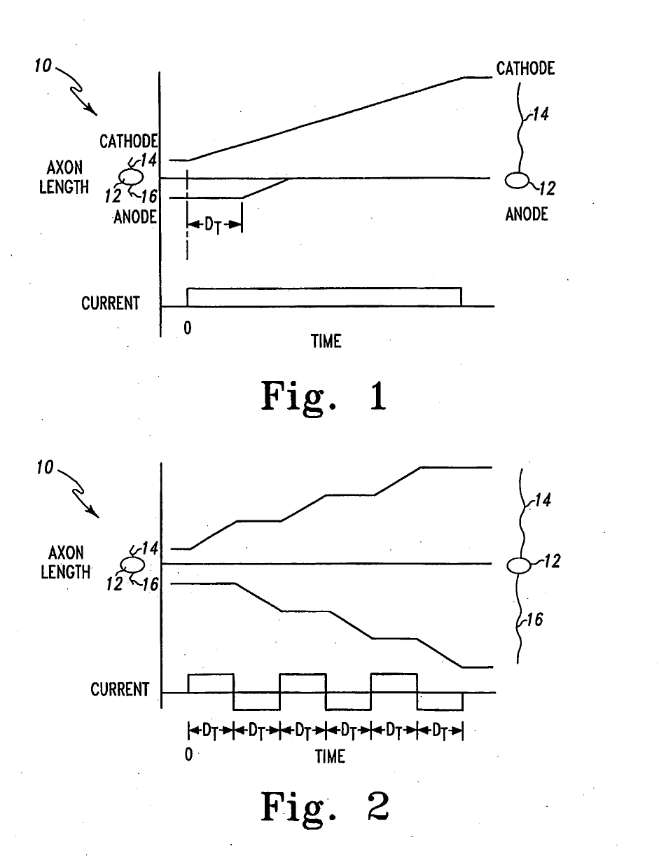

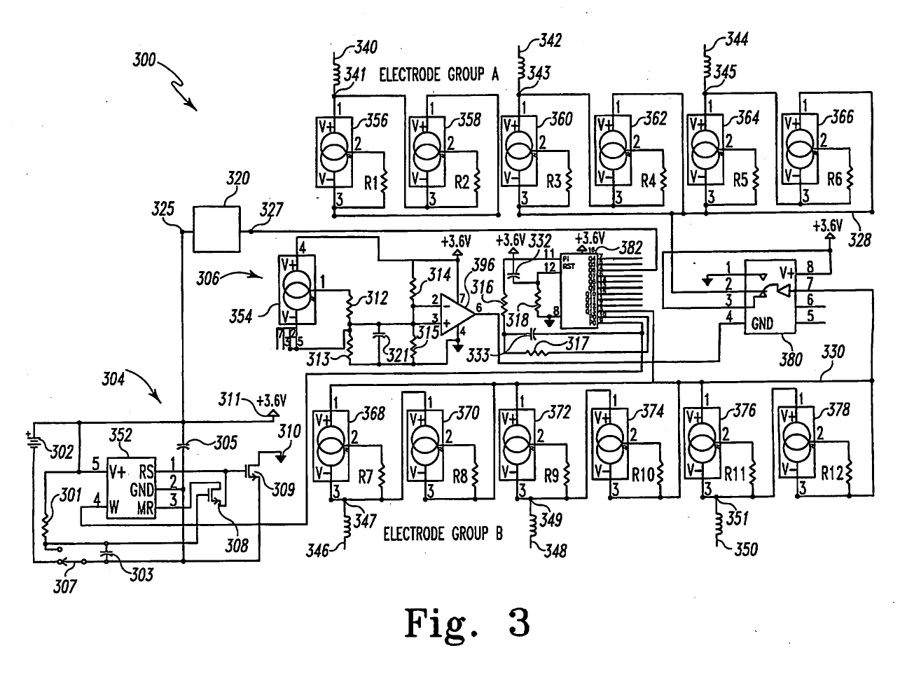

FIGS. 1 and 2 show the effects on axon growth of an applied steady state DC electrical field (FIG. 1) and by an applied oscillating electrical field (FIG. 2). Referring to FIG. 1, a nerve cell 10 is shown at the left-hand side of FIG. 1 having a cell body or soma 12 from which an axon 14 extends upwardly and an axon 16 extends downwardly. At time 0, a constant current DC stimulus is applied to the nerve cell 10 such that axon 14 will be extending toward the cathode or negative pole of a DC stimulus signal and axon 16 will be extending toward the anode or positive pole of the DC stimulus. Axon 14 begins to grow almost immediately. However, after a period of time, i.e., the “die back period” (D_{T}), reabsorption of the anodally facing axon 16 into the cell body 12 begins. Eventually after a sufficient time of continually facing the anode, axon 16 will be completely reabsorbed into cell body 12. At the right-hand side of FIG. 1 for illustration purposes nerve cell 10 is shown wherein axon 14 has grown substantially longer but axon 16 has been reabsorbed into cell body 12.

In FIG. 2, the same reference numbers will be used to identify the elements of FIG. 2 which correspond to elements of FIG. 1. Nerve cell 10 is shown at the left-hand side of FIG. 2 having a cell body 12, an upwardly extending axon 14 and a downwardly extending axon 16. At time 0, a constant current DC stimulus is applied to nerve cell 10 such that axon 14 is extending toward the cathode and axon 16 is extending toward the anode of the DC stimulus. After a predetermined period of time, the polarity of the DC stimulus is reversed. Axon 14 will now be extending toward the anode and axon 16 will be extending toward the cathode of the DC stimulus. The predetermined period of time is selected to be less than the die back period (D_{T}) of the anodal facing axon. Significant die back of anodal facing axons begins to occur about one hour after the DC stimulus is applied but die back may begin sooner or later. Therefore, the predetermined period should not exceed one hour. As shown in FIG. 2, an oscillating DC field stimulates growth of the axons facing both direction. This is due to the fact that growth of cathodal facing axons is stimulated almost immediately after the DC stimulus is applied but die back of the anodal facing axons does not become significant until after the die back period elapses. Since the polarity of the DC stimulus is switched before the die back period elapses, growth of axons in both directions is stimulated with the result that axons 14, 16 of nerve cell 12 both grow significantly longer as shown at the right-hand side of FIG. 2.

In accordance with the present disclosure, the nerves in the central nervous system of a mammal are stimulated to regenerate by applying an oscillating electrical field to the central nervous system. The oscillating electrical field is a constant current DC stimulus which is first applied in one direction for a predetermined period of time, and then applied in the opposite direction for the predetermined period of time. In other words, the polarity of the constant current DC stimulus is reversed after each predetermined period of time. The predetermined period of time is selected to be less than the die back period of anodal facing axons, but long enough to stimulate growth of cathodal facing axons. This predetermined period will be termed the “polarity reversal period” of the oscillating electrical field. In one disclosed embodiment, this polarity reversal period is between about thirty seconds and about sixty minutes.

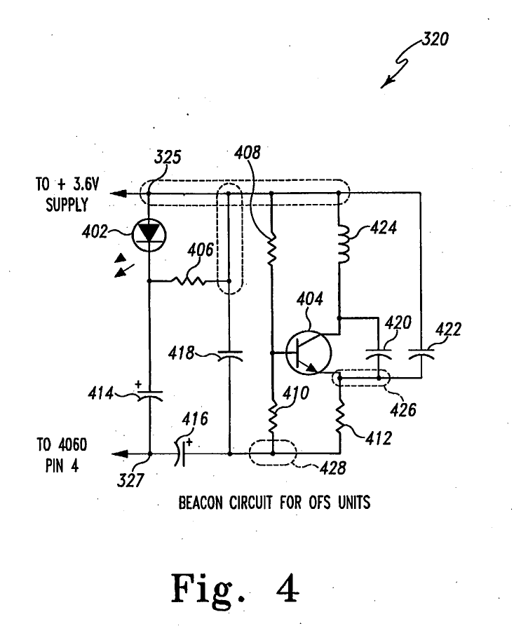

FIG. 3 shows a schematic of circuit 300 according to one disclosed embodiment of a device for generating an oscillating electrical field for stimulating nerve regeneration. Circuit 300 comprises electronic components electrically interconnected as shown in FIG. 3. Conventional symbols are used to denote the components. Circuit 300 as shown in FIG. 3 comprises electrodes 340, 342, 344, 346, 348, and 350; processor supervisory circuit 352; adjustable current sources 354, 356, 358, 360, 362, 364, 366, 368, 370, 372, 374, 376, and 378; switch 380; and timer 382. Circuit 300 as shown in FIG. 3 also comprises optional beacon circuit 320, electrically interconnected between nodes 325 and 327. Electrode 340 is coupled to the output terminal 341 of the back-to-back adjustable current sources 356 and 358 which constitute a portion of the DC stimulus generator. Electrode 342 is coupled to the output terminal 343 of the back-to-back adjustable current sources 360 and 362 which constitute a portion of the DC stimulus generator. Electrode 344 is coupled to the output terminal 345 of the back-to-back adjustable current sources 364 and 366 which constitute a portion of the DC stimulus generator. Electrode 346 is coupled to the output terminal 347 of the back-to-back adjustable current sources 368 and 370 which constitute a portion of the DC stimulus generator. Electrode 348 is coupled to the output terminal 349 of the back-to-back adjustable current sources 372 and 374 which constitute a portion of the DC stimulus generator. Electrode 350 is coupled to the output terminal 351 of the back-to-back adjustable current sources 376 and 378 which constitute a portion of the DC stimulus generator. Electrodes 340, 342, and 344 comprise Electrode Group A and thus output terminals 341, 343 and 345 constitute one group of output terminals. Electrodes 346, 348, and 350 comprise Electrode Group B and thus output terminals 347, 349 and 351 constitute another group of output terminals.

Circuit 300 includes a power supply and supervisory section 304, and a secondary watchdog section 306. The power supply and supervisory section 304 produces a 3.6 volt supply for powering the remaining devices of circuit 300, including secondary watchdog section 306 and the optional beacon circuit 320 and the main oscillator of timer 382. Additionally, the power supply and supervisory section 306 supervises the oscillator circuitry of the timer 382 to determine if there is failure of the oscillator circuit.

The power supply and supervisory circuit 304 includes a battery 302, processor supervisor circuit 352, a resistor 301, a first capacitor 303, a second capacitor 305, a switch 307, a first transistor 308, and a second transistor 309 configured as shown in FIG. 3 to provide a 3.6 volt potential between a ground terminal 310 and a positive voltage terminal 311 for so long as the oscillator circuitry of the timer 382 is operating within desired parameters as explained in greater detail below. In one illustrated embodiment, the battery 302 may be a 3.6v Tadiran TL-5903 battery although other batteries, including, but not limited to, rechargeable batteries, e.g. rechargeable battery 802, may be used within the scope of the disclosure.

In one illustrated embodiment, the switch 307 may be an HSR-502RT reed switch available from Hermetic Switch, Inc., Chickasha, Okla. However, other switches may be used within the scope of the disclosure. The HSR-502 reed switch is a single pole-double throw (SPDT) switch enclosed in a glass capsule.

In one illustrated embodiment, transistors 308 and 309 may be BSS138 transistors available from Fairchild Semiconductor Corporation, South Portland, Me., although other transistors and appropriate components can be used within the scope of the disclosure. In one illustrated embodiment, the transistors 308, 309 are N-Channel Logic Level Enhancement Mode Field Effect Transistors. The values of the resistor 301 and capacitors 303, 305 are chosen as required to meet design parameters. In the illustrated embodiment, resistor 301 is a 1 Mohm resistor and capacitors 303, 305 are 0.047 microfarad capacitors.

The processor supervisor circuit 352 receives a clock pulse signal from the oscillator section of timer 382. In one illustrated embodiment, the processor supervisor circuit 352 is a TPS 3823 Processor supervisor circuit with watchdog timer input (W) and Manual Reset Input (/MR) available from Texas Instruments, Dallas Tex. The illustrated processor supervisor circuit 352 includes a Power-On Reset Generator With Fixed Delay Time of 200 ms. The illustrated processor supervisor circuit 352 provides circuit initialization and timing supervision for the timer 382. During power-on, /RESET (/RS) is asserted when supply voltage (V+) becomes higher than 1.1 V. Thereafter, the supply voltage supervisor monitors the supply voltage and keeps /RESET active as long as the supply voltage remains below the threshold voltage. An internal timer delays the return of the output to the inactive state (high) to ensure proper system reset. The delay time, td, starts after supply voltage has risen above the threshold voltage. When the supply voltage drops below the threshold voltage, the output becomes active (low) again. The illustrated processor supervisory circuit 352 has a fixed-sense threshold voltage set by an internal voltage divider. The illustrated processor supervisor circuit 352 incorporates a manual reset input, (/MR). A low level at the manual reset input (/MR) causes /RESET to become active. The illustrated processor supervisor circuit 352 includes a high-level output at /RESET (/RS).

The arrangement illustrated in FIG. 3 is configured so that when a low level is received on the /Reset pin of the processor supervisor circuit 352, the gate of the FET 308 receives no current effectively shutting down FET 309. When FET 309 is shut down, the power supply is effectively shut down causing the remaining components of the circuit 300 to be without power. Once FET 309 is shut down, transistor 308 asserts a low signal on the /MR pin of the supervisor circuit 352 effectively locking down the circuit until the power is cycled utilizing switch 307. This configuration of timer 382, supervisory circuit 352 and FETs 308, 309 acts as a failsafe device to shut down the oscillating field circuit whenever there is an apparent failure of the oscillator of the timer 382 so that the axons facing anodes will not be subjected to a current beyond the beginning of the die back period. The illustrated processor supervisor circuit 352 includes watchdog timer that is periodically triggered by a positive or negative transition at the watchdog timer input (W). The watchdog timer receives the clock pulse from the timer 382 of the secondary watchdog section 306. When the supervising system fails to retrigger the watchdog circuit within the time-out interval, tₜₒᵤₜ, /RESET becomes active which, as described above shuts down FET 309 and causes FET 308 to assert a low signal on the /MR pin of the process supervisor circuit. This event also locks down and removes power from all of the other components of the circuit 300 (except battery 302) until power is cycled via switch 307. The positive terminal of the battery 302 is electrically connected to the supply voltage input (V+) of the processor supervisory circuit 352, one terminal of resistor 301, the positive electrode of the second capacitor 305 and to the positive output terminal 311. The second terminal of the resistor 301 is electrically connected to a node electrically connected to one terminal of the switch 307, the positive electrode of the first capacitor 303 and the gate of the reset transistor 308 of the above described power-on/reset delay network. The second terminal of the switch 307 is electrically connected to the negative terminal of the battery 302. The pole of the switch 307 is electrically connected to a node electrically connected to the negative electrode of the first capacitor 303, the ground pin (GND) of the processor supervisor circuit 352, the negative electrode of the second capacitor 305 and the source of the second transistor 309. The gate of the second transistor 309 is coupled to a node coupled to the /RESET pin (/RS) and the source of the first transistor 308. The drain of the second transistor 309 is coupled to the ground terminal 310. The drain of the first transistor is coupled to the manual reset pin (/MR) of the processor supervisor circuit 352. The watchdog timer input (W) of the processor supervisor circuit 352 is coupled to the PO pin of the timer 382.

The secondary watchdog section 306 includes adjustable current supply 354, switch 380, op amp 396, resistors 312-315 and capacitors 321. While the illustrated secondary watchdog section 306 is configured in accordance with the schematic shown in FIG. 3, it is within the scope of the disclosure for the secondary watchdog section 306 to be configured using other or additional components or for the section to be implemented on a single or multiple integrated circuits or a portion of a single or multiple integrated circuits implementing circuit 300.

In one illustrated embodiment, op amp 386 is an Analog Devices OP90GS Precision, Low Voltage Micropower Operational Amplifier, available from One Technology Way, Norwood, Mass. Other operational amplifiers or amplifier circuitry may be utilized within the scope of the disclosure.

In one illustrated embodiment, the switch 380 is a MAX4544CSA Low-Voltage, Single-Supply Dual SPDT Analog Switch available from Maxim Integrated Products, Sunnyvale, Calif. The MAX4544 is a dual analog switch designed to operate from a single voltage supply, which because of its low power consumption (5 μW) is particularly well adapted for battery-powered equipment. The disclosed switch 380 offers low leakage currents (100 pA max) and fast switching speeds (tON=150 ns max, tOFF=100 ns max). The MAX4544 switch 380 is a single pole/double-throw (SPDT) device.

In one illustrated embodiment, the timer 382 is a CD4060B type CMOS 14-stage ripple-carry binary counter/divider and oscillator, available from Texas Instruments, Dallas, Tex. The illustrated CD4060B timer 382 consists of an oscillator section and 14 ripple-carry binary counter stages. A RESET input is provided which resets the counter to the all-O's state and disables the oscillator. A high level on the RESET line accomplishes the reset function. All counter stages are master-slave flip-flops. The state of the counter is advanced one step in binary order on the negative transition of PI (and PO). All inputs and outputs are fully buffered. Schmitt trigger action on the input-pulse line permits unlimited input-pulse rise and fall times.

In the illustrated embodiment, the watchdog timer input to the processor supervisor circuit 352 is coupled to the PO output of the timer 382 to provide a pulsed clock signal to indicate proper operation of the timer 382 which controls the polarity reversal period. Absence of this signal causes the supervisor circuit 352 to shut down power to the entire system. The /PO pin of the timer 382 is coupled through resistors 316 and 317 to the PI pin of the timer 382. The positive electrode of capacitor 323 is coupled to a node coupling the terminals of resistors 316 and 317, while the negative electrode of the capacitor 323 is coupled to a node coupled to the PO pin of the timer 382 thereby forming a free running oscillator. The period of the free-running oscillator is determined by the values of the resistors 316 and 317 and the capacitor 323. In the illustrated embodiment, the resistors 316 and 317 each have a resistance of 1 Mohm and the capacitor has a 0.047 micro-farad capacitance so that the oscillator runs at a frequency to generate the desired reversal period. The values of the resistors 316 and 317 and capacitor 323 can be varied to obtain reversal periods of different values within the scope of the disclosure.

The Q7 pin of the counter of the timer is coupled to node 327 to provide a pulse to activate the optional beacon circuit 320. The Q14 pin of the timer 382 is coupled to a group B node 330, i.e. a node providing power to the adjustable current sources 368, 370, 372, 374, 376 and 378 driving the Group B electrodes 346, 348 and 350. The reset pin of the timer 382 is coupled to a node that is coupled through the capacitor 322 to the positive voltage terminal 311 and coupled through resistor 318 to a node coupled to both the ground terminal 310 and the ground pin of the timer 382. The power supply pin of the timer 382 is coupled to the positive voltage terminal 311.

The adjustable current source 354 of the secondary watchdog section 306 has its positive supply pin (V+) coupled to a node coupled to the positive voltage terminal 311. This adjustable current source 354 provides a reference current that is utilized by op amp 396 to generate a signal to turn off the output power when the voltage drops below a specified value (illustratively 2.8V). In the illustrated embodiment, the adjustable current source 354 was selected to generate a second reference voltage instead of selecting a zenor diode to avoid the power loss associated with zenor diodes when utilized as reference voltage generators. The output power is interrupted in the illustrated circuit 300 by adjustable current source 354 and op amp 396 cooperating to lift the ground of switch 380 to interrupt current outflow to the group A electrodes.

The negative pin (V−) of the adjustable current source 354 is coupled to the central node of a first voltage divider formed by resistors 312 and 313. The central node of the first voltage divider is coupled through the resistor 313 to the ground terminal 310 and is also coupled through a node to the non-inverting input of op amp 396. The capacitor 321 is in parallel with the resistor 313 between the central node of the first voltage divider and the ground terminal 310. The resistors 314 and 315 form a second voltage divider having a central node coupled to the inverting input of the op amp 396. The second voltage divider is coupled between the positive voltage terminal 311 and the ground terminal 310. The positive voltage terminal 311 is also coupled to the voltage supply pin of the op amp 396 and the ground terminal 310 is coupled to the ground pin of the op amp 396. The output of the op amp is coupled to the Ground-Negative Supply Input pin of the switch 380.

The Positive Supply Voltage Input pin of the switch 380 is coupled to the positive voltage terminal 310. The Ground-Negative Supply Input pin of the switch 380 is coupled to the output of the op amp 396. The Normally Open pin of the switch 380 is coupled to the ground terminal 310. The Common pin of the switch 380 is coupled to the Group A node, i.e. the node for providing the power to the adjustable current supplies 356, 358, 360, 362, 364 and 366 powering the Group A electrodes 340, 342, 344. The Normally Closed pin of the switch 380 is coupled to the positive voltage terminal 311. The Digital Control Input pin of the switch 380 is coupled to the Group B node which, as mentioned above, is also coupled to the Q14 pin of the timer 382. Thus, the timer 382 is configured to cause the Group A electrodes and Group B electrodes to switch between anodes and cathodes to generate a waveform such as that shown in FIG. 2.

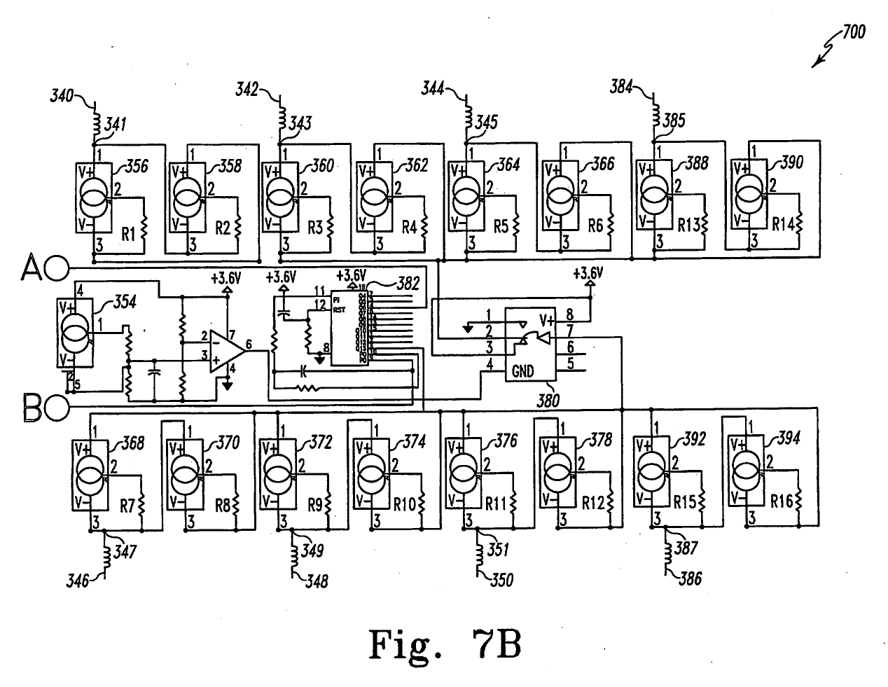

FIGS. 7A and 7B (which together make up FIG. 7) show a schematic of an alternative circuit 700 for generating an oscillating electrical field for stimulating nerve regeneration. The circuit 700 is substantially similar to circuit 300 and thus the same reference numerals are utilized for identical or similar components. Circuit 700 differs from circuit 300 in that circuit 700 provides four electrodes in each electrode group A and B whereas circuit 300 provides only three electrodes in each electrode group A and B. Thus, circuit 700 includes two additional electrodes 384 and 386, one of which, electrode 384 is in electrode group A and one of which, electrode 386, is in electrode group B. In circuit 700, electrodes 340, 342, 344 and 384 comprise Electrode Group A and electrodes 346, 348, 350 and 386 comprise Electrode Group B. Circuit 700 also includes four additional adjustable current sources, 388, 390, 392 and 394, two of which, adjustable current sources 388 and 390, are connected in parallel with opposite polarity to supply bidirectional current through output terminal 385 to electrode 384 and two of which, adjustable current sources 392 and 394, are connected in parallel with opposite polarity to provide bidirectional current through output terminal 387 to electrode 386. Otherwise, the description herein of circuit 300 is equally applicable to circuit 700 and shall not be repeated with respect to circuit 700. The circuit 700 is particularly suitable for facilitating the provision of a substantially uniform electrical field of a desired strength imposed over about 10 cm to 20 cm of damaged spinal cord as described in greater detail below.

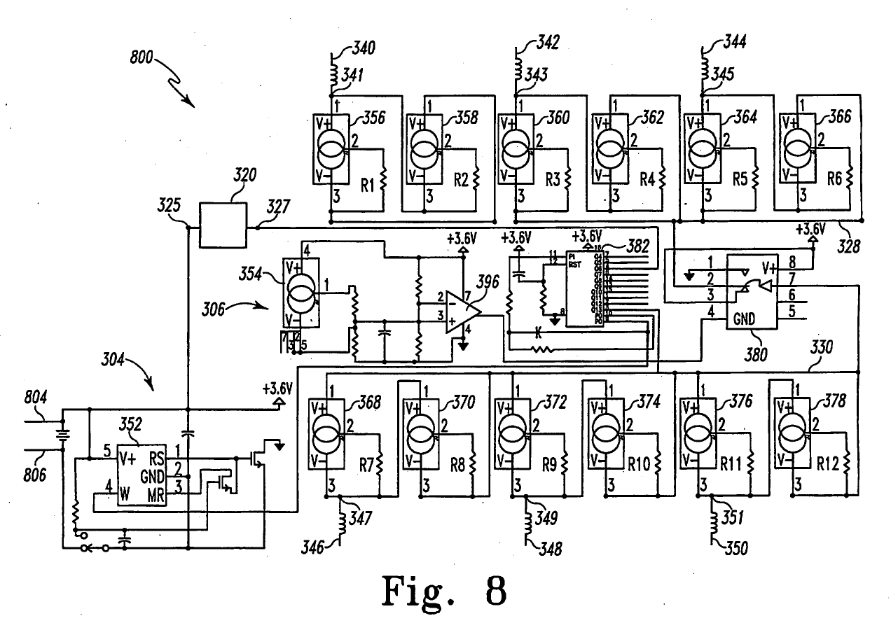

FIG. 8 shows a detail of a schematic of an embodiment of a rechargeable circuit 800 for generating an oscillating electrical field that is very similar to circuit 300 shown in FIG. 3. Because circuit 800 is so similar to circuit 300, identical reference numerals shall be utilized to identify identical components and the description of the identical components will not be repeated with regard to circuit 800, it being understood that the description of those components with regard to circuit 300 is equally applicable to circuit 800.

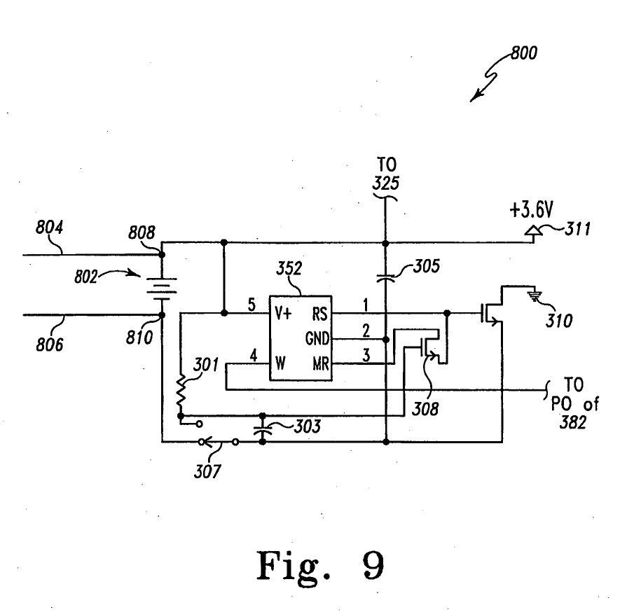

Circuit 800 does differ however in some respects from circuit 300, specifically, as shown, for example, in FIG. 8 and in greater detail in FIG. 9, a rechargeable charge storage device 802, and recharging electrodes 804 and 806 are provided in circuit 800 that replace the battery 302 of circuit 300. Recharging electrodes 804 and 806 are coupled respectively to nodes 808 and 810 of circuit 800. In this embodiment the rechargeable charge storage device 802 is preferably a rechargeable battery, and may comprise a lithium ion (Li-Ion), nickel metal hydride (NiMH) cell, nickel-cadmium (NiCad) cell, or any other available rechargeable cells or combination of cells.

In this embodiment, the recharging electrodes 804 and 806 are implanted near the surface of skin of the patient. In one preferred embodiment, the recharging electrodes 804 and 806 are implanted into the dermis, either in the papillary layer, or the reticular layer. In another preferred embodiment, the recharging electrodes 804 and 806 are implanted in the epidermis, in either the stratum spinosum or stratum basale layer. Implantation of the recharging electrodes 804 and 806 in the stratum corneum is also possible, but could cause discomfort or other problems because of the near proximity of the recharging electrodes 804 and 806 to the surface.

In operation, an external charging circuit (not shown) is removably coupled to the recharging electrodes 804 and 806 prior to implantation of the circuit in the patient. Preferably, the external charging circuit is removably coupled to the recharging electrodes 804 and 806 for a sufficient period of time to fully charge the rechargeable charge storage device 802 just prior to a procedure to implant the circuit. After some period of time, six weeks for example, the rechargeable battery 802 may discharge to the point that the circuit is no longer operating at an optimum level. At this time, or any time, a simple procedure may be performed under local anesthetic to expose the recharging electrodes 804 and 806. During this procedure, the external charging circuit may be removably coupled to the recharging electrodes 804 and 806 for a period of time in order to recharge the rechargeable charge storage device 802. Once the recharging of the rechargeable charge storage device 802 is complete, the recharging electrodes 804 and 806 may be re-implanted into the patient.

In the illustrated embodiments of circuits 300, 700 and 800 each electrode 340, 342, 344, 346, 348, 350, 384 and 386 is coupled to a pair of adjustable current sources connected in parallel with opposite polarity to generate the desired bidirectional current (ISET) for the electrode. In the illustrated embodiment, electrode 340 is coupled to current sources 356 and 358, electrode 342 is coupled to current sources 360 and 362, electrode 344 is coupled to current sources 364 and 366, electrode 346 is coupled to current sources 368 and 370, electrode 348 is coupled to current sources 372 and 374 and electrode 350 is coupled to current sources 376 and 378. In the illustrated embodiment, since each current source provides current in one direction only, i.e. uni-directional current, identical mirrored current sources are connected in parallel with opposite polarity (also referred to as “back-to-back”) to provide bidirectional current to facilitate the switching of the polarity of the groups of electrodes as described herein. The bias current for each first adjustable current source is determined in part by the values of bias resistors R1-16. The arrangement of the current sources in parallel with opposite polarity facilitates bidirectional current flow through the electrodes. While illustrated as utilizing back-to-back adjustable current sources to provide the power to electrodes, it is within the scope of the disclosure for other current sources, including, but not limited to, stand alone bidirectional adjustable current sources, to be utilized to provide power to the electrodes in circuits 300, 700 and 800.

Among the current sources that can be utilized for current sources 354, 356, 358, 360, 362, 364, 366, 368, 370, 372, 374, 376, 378, 388, 390, 392 and 394 are the LM334 series of three terminal adjustable current source available from National Semiconductor. The total current through each LM334 (ISET) is the sum of the current going through the SET resistor (in the illustrated embodiment resistors R1-16) and the LM334's bias current (IBIAS). Other current sources can be utilized in circuits 300, 700 and 800 within the scope of the disclosure and calibrated to produce the desired output current to each electrode.

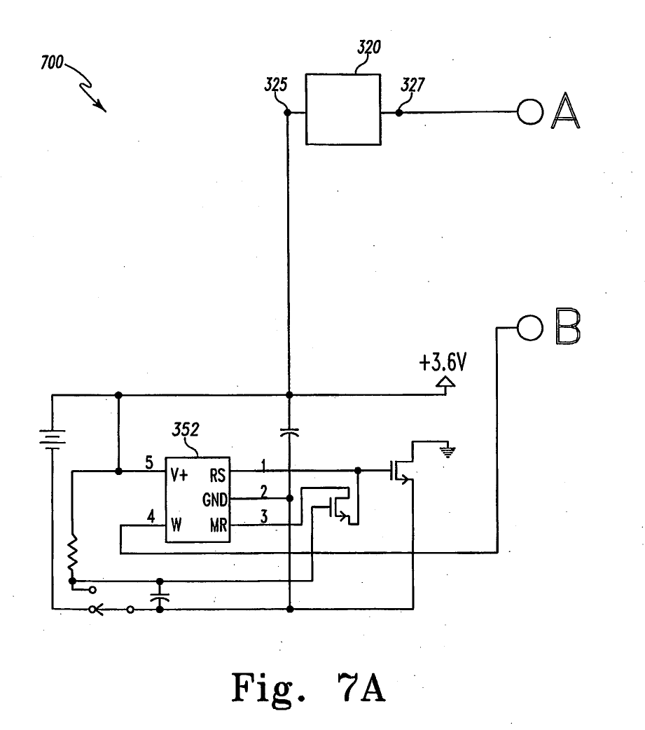

Referring now to FIG. 6, there is shown a schematic of circuit 600 for generating an oscillating electrical field for stimulating nerve regeneration. Circuit 600 is particularly suitable for use in small mammals because the components utilized are somewhat smaller than those utilized in Circuits 300, 700 and 800. Circuit 600 comprises electronic components electrically interconnected as shown in FIG. 6. Circuit 600 includes a constant DC power supply section 601 and an oscillating signal generation section 603. It is within the scope of the disclosure for those portions of circuits 300, 700 and 800 that generate a constant DC voltage to be substituted for the constant DC power supply section 601 of circuit 600. The portions of circuits 300, 700 and 800 that generate a constant DC voltage are the power supply and supervisory section 304.

Referring now to FIG. 6, conventional symbols are used to denote the components. Circuit 600 as shown in FIG. 6 comprises counter 602, switch 604, JFETs 606 and 608, and electrodes 610 and 612, diodes 614 and 616, NAND Gate 618, Jumpers 620 and 622, batteries 624, 626, switch 628, loop 630, capacitors 632, 634 and resistors 636, 638, 640, 642 and 644. While illustrated as single electrodes, electrode 610 is representative of one or more group B electrodes (e.g. electrodes 346, 348, 350 and 386 of circuits 300, 700 and 900) and electrode 612 is representative of one or more group A electrodes (e.g. electrodes 340, 342, 344 and 384 of circuits 300, 700 and 900).

The switch 604 is a 74LVC1G66 Bilateral switch available from Philips Semiconductors, Eindhoven, The Netherlands.

The 74LVC1G66 is a high-speed Si-gate CMOS device. The 74LVC1G66 provides an analog switch. The switch has two input/output pins (Y and Z) and an active HIGH enable input pin (E). When pin E is LOW, the analog switch is turned off.

JFETs 606 and 608 are N-Channel Silicon Junction Field-Effect Transistor available from InterFET Corporation, Garland Tex.

In one illustrated embodiment, the counter 602, like timer 382 in circuits 300, 700 and 800, is a CD4060B type CMOS 14-stage ripple-carry binary counter/divider and oscillator, available from Texas Instruments, Dallas, Tex. With this and the prior statements regarding circuit 600 in mind, it will be seen that the pins of counter 602 are configured similarly to the pins in timer 382 in circuits 300, 700 and 800. However counter 602 is also coupled to a chopper circuit as explained below. Due to the similarity of the configuration of counter 602 in circuit 600 and timer 382 in circuits 300, 700, 800, it is easily understood how circuits 300, 700 and 800 can be modified to implement a chopper circuit.

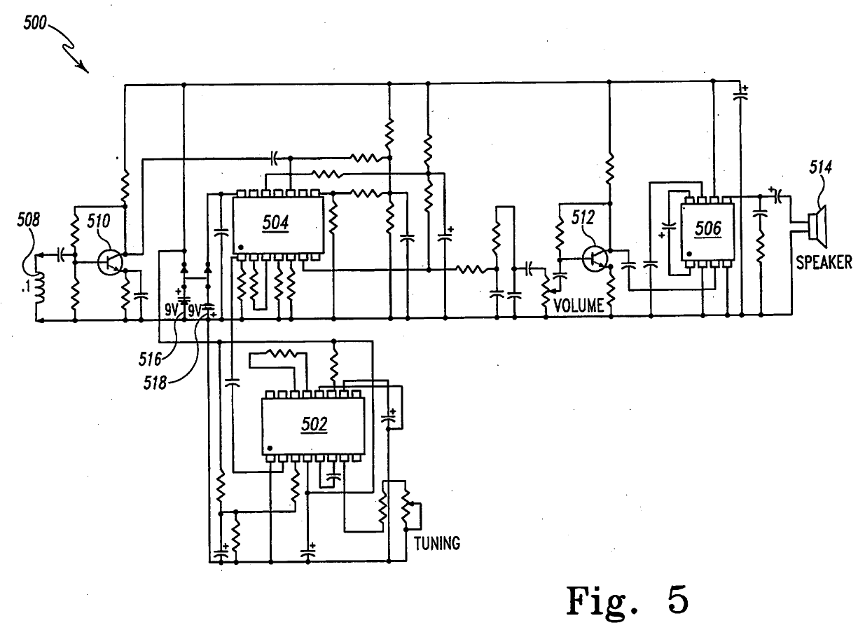

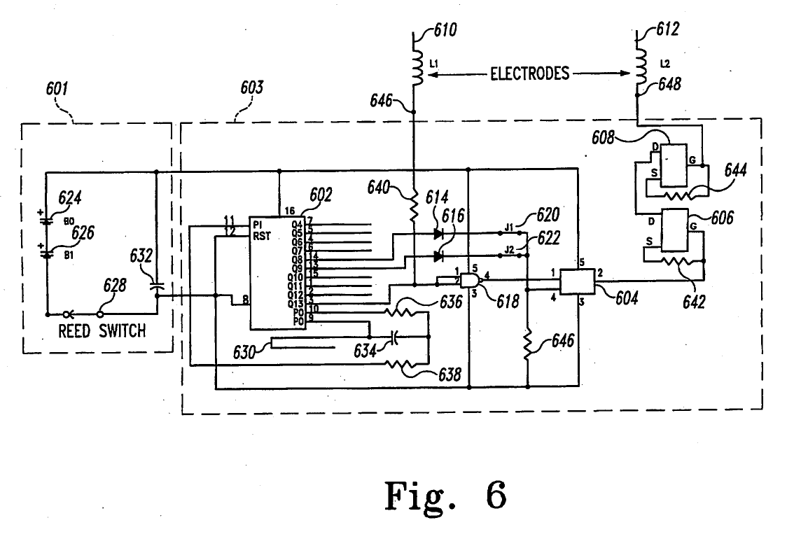

In the illustrated embodiment, loop 630 consists of a simple loop of wire. Since circuit 600 is configured for use in small mammals, a complex beacon circuit 320, such as that shown in FIG. 4, might not be suitable for utilization with the circuit 600 when it is implanted into a small mammal. The oscillator of the counter 602 produces electronic noise (illustratively at approximately 11 Hertz) that is present on the PO pin. Thus, when loop 630 is coupled to the PO pin, an electrical field is generated of sufficient strength to be detected up to about a half an inch from the circuit 600. This electrical field can be detected by an ordinary portable audio amplifier with an unshielded piece of wire connected to the input or by a receiver such as that illustrated in FIG. 5. Thus, proper operation of the circuit 600 can be verified either before or after implantation of circuit 600 into a mammal by detecting the signal radiated by loop 630.

The /PO pin of the counter 602 is coupled through resistors 636 and 638 to the PI pin of the counter 602. The negative electrode of capacitor 624 is coupled to a node coupling the terminals of resistors 636 and 638, while the positive electrode of the capacitor 634 is coupled to a node coupled to the PO pin of the counter 602 and the loop 630. The Q7 pin of the counter 602 in circuit 600 is shown as floating, but it is within the scope of the disclosure for the Q7 pin of the counter 602 to be coupled to node 327 to provide a pulse to activate the optional beacon circuit 320.

The Q14 pin of the counter 602 is coupled through a node coupled through resistor 640 to a group B node 646, i.e. a node providing power to the Group B electrode 610, and to the logic inputs of the NAND gate 618. The reset pin of the counter 602 is coupled to a node that is coupled through the capacitor 632 to the positive terminal of battery 624 and coupled to a node coupled to the ground pin of the counter 602 and through the switch 628 to the negative terminal of battery 626. The power supply pin of the counter 602 is coupled to the positive terminal 311 of battery 624. Batteries 626 and 624 are coupled in series.

The Q8 pin of counter 602 is coupled to the anode of diode 614, the cathode of diode 614 is coupled to one terminal of jumper 620. The other terminal of jumper 620 is coupled to a node coupled to Enable input pin of the switch 604, to one terminal of jumper 622 and through resistor 646 and switch 628 to the negative terminal of battery 626. The other terminal of jumper 622 is coupled to the cathode of diode 616 which has its anode coupled to the Q9 pin of the counter 602. The Y independent input/output pin of switch 604 is coupled to the output of the NAND gate 618. The Z independent output/input pin of switch 604 is coupled to a node that is coupled to the gate of JFET 606 and through resistor 642 to the source of JFET 606. The drain of JFET 608 is coupled to the drain of JFET 608. The source of JFET 608 is coupled through resistor 644 to a node coupled to the gate of JFET 608 and to A electrode power node 648.

In the illustrated embodiment of circuit 600, JFETs 606 and 608 and their associated resistors 642 and 644, respectively, comprise bidirectional constant current sources. JFETs 606 and 608 and their associated resistors 642 and 644 are utilized as constant current sources in circuit 600 instead of the adjustable current sources found in circuits 300, 700 and 800, because they reduce the size of circuit 600 to facilitate implantation of circuit 600 into small mammals.

The ground pin of NAND gate 618 and the ground pin of switch 604 are coupled through switch 628 to the negative terminal of battery 626. The supply voltage pin of NAND gate 618 and the supply voltage pin of switch 604 are coupled to the positive terminal of battery 624

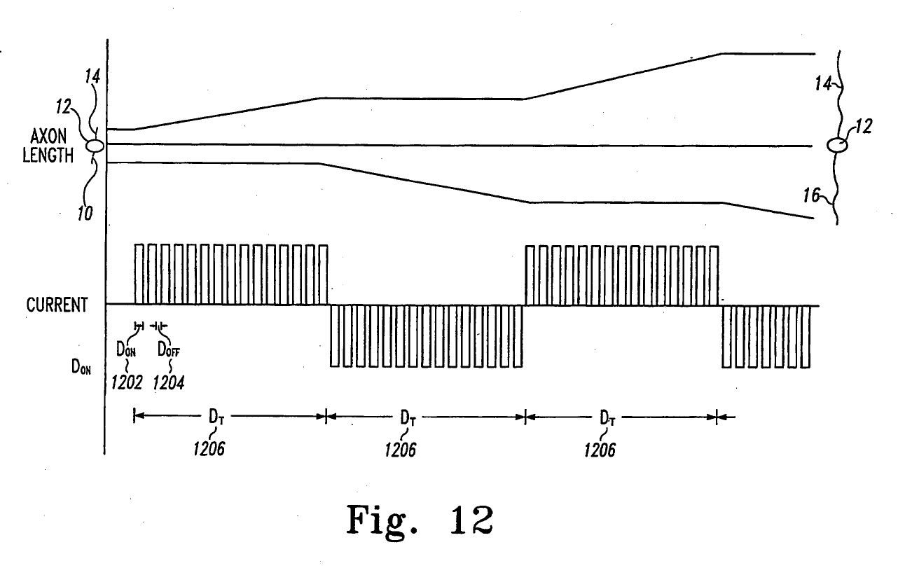

Circuit 600 comprises a current chopping circuit. The DC current is “chopped” or turn off for a short but fixed amount of time. For example, by setting jumper 620 to a 25% duty cycle and jumper 622 to a 50% duty cycle, the DC current exhibits an on duty cycle Don 1202 of 75% (jumper 620 plus jumper 622) and off duty cycle Doff 1204 for 25% of the time, chopped once per minute producing a wave form as shown in FIG. 12. If this amount of time is small enough compared to the overall time, the nerve cell regeneration continues at the same rate as if the current were held steady. However, chopping the DC current in the manner increases battery life, or enables the battery to power other device functions while maintaining a lifespan sufficient for regeneration to be substantially completed. Additionally, punctuated, pulsatile or discontinuous oscillating DC electric fields are believed to work as well, if not, in some case when utilized to heal certain types of nerves, better than, constant oscillating DC electric fields. Thus, there is the expectation that the chopping circuit will generate a pulsatile electric field that may improve functional recovery as well as save battery life.

In one disclosed embodiment, where polarity reversal period DT 1206 of the oscillating electrical field is set to 10 minutes and the duty cycle of the current is set to 75%, circuit 600 produces an output wave form as shown in FIG. 12. It is within the scope of the disclosure for the polarity reversal period to be between about thirty seconds and about sixty minutes. It is also within the scope of the disclosure for the polarity reversal period to be between a minimal clinically effective value to stimulate nerve regeneration in the cathode-facing axon and a value less than the beginning of the die-back period in the anode-facing axon. Clinically effective results can readily be obtained when the reversal period is set between ten and twenty minutes. Highly effective clinical results have been achieved with the duty cycle set to approximately fifteen minutes. It is also within the scope of the disclosure, though not preferred because regeneration of axons induced to die back through the area of die back will be required before therapeutic growth will be induced, for the polarity reversal period to exceed the beginning of the die back period but be less than the time for die back to proceed to the point of killing the nerve cell.

It is within the scope of the disclosure for the on duty cycle 1202 to be between 60% and 99%. Clinically effective results may be obtained in one embodiment when the on duty cycle 1202 is between 70% and 85%. Clinically effective results may be obtained in another embodiment when the on duty cycle 1202 is between 75% and 80%.

In operation, a device comprising circuit 300, 600, 700 or 800 is implanted into an injured mammal shortly after the time of central nervous system injury. The device comprising circuit 300, 600, 700, 800 remains implanted for a period of time post-injury. For example, the device comprising circuit 300, 600, 700, 800 remains implanted for up to fourteen weeks in humans.

Power is applied to the device comprising circuit 300, 600, 700, 800 during implantation. When power is applied, the circuit generates an oscillating electrical field at Electrode Group A and Electrode Group B. That is, the circuit generates a constant current DC stimulus the polarity of which is reversed periodically after the expiration of a predetermined period of time determined by the operation of timer 382 (or counter 602 in circuit 600). Electrode Group A and Electrode Group B alternately comprise cathode and anode terminals, respectively, depending upon the polarity of the DC stimulus.

The voltage between from Electrode Group A and Electrode Group B is selected to provide sufficient field strength in the section of the spinal cord in which nerve regeneration is to be stimulated. A field strength of 200 μV/mm in the spinal cord adjacent the lesion will stimulate regeneration. The current needed to achieve this field strength is determined by the geometry of the animal in which a device comprising circuit 300, 600, 700, 800 is used and the location of the nearest electrode to the lesion. While a field strength of 200 μV/mm will stimulate regeneration, a field strength of 600 μV/mm has been found to produce clinically effective nerve regeneration.

Illustratively, electrodes 340, 342, 344, 346, 348, and 350 comprise silastic insulated platinum-iridium electrodes. Electrode Groups A and B are implanted on opposite sides of a lesion in the spinal cord. It is sufficient to implant Electrode Groups A and B in a laminectomy adjacent the spinal cord but not actually in the spinal cord. Further, moving the anode from within the laminectomy to a site on the muscle dorsal to the same area results in only about a ten percent drop in field strength as does the converse of moving the cathode to a more superficial position while leaving the anode in the laminectomy. Further, uniform field homogeneity can be achieved by locating the electrodes anywhere on the midline of the spinal cord, including locating both electrodes on the same side of the lesion but spaced apart, although locating the electrodes on opposite sides of the lesion is preferred.

Applicants have also found that the field strength within the spinal cord at the site of the lesion depends upon the location of the current delivery electrodes. The convergence of current to an electrode produces high current density and hence higher field strength near each electrode. The closer one electrode is to the lesion site, the less critical is the placement on the other to maintain high field strengths. However, as a current delivery electrode location approaches the location of the lesion, current direction becomes less uniform. At a lesion exactly half-way between two electrodes placed on the midline, the current will all be oriented along the long axis of the subject animal. As one of the electrodes is moved closer to the lesion, there will be a larger vertical (dorsal-ventrical) component of the current at the lesion (assuming that the electrodes remain a few millimeters dorsal to the target tissue).

As a compromise between uniform current direction and maximum field strength, applicants have chosen to position the electrodes two vertebral segments on either side of the lesion in their spinal cord studies. In the guinea pig studies applicants have conducted, it appears that at least one electrode should be positioned within one convergence zone of an electrode from the lesion. A convergence zone is that area in which the current convergence to the electrode so dominates the field strength that the position of the other electrode is relatively inconsequential. Utilizing the illustrated electrodes, the convergence one is approximately 1 cm. Therefore, by placing one electrode within 1 cm of the lesion, the position of the other becomes relatively inconsequential and becomes a matter of convenience. It should be noted, however, that the electrodes can be located further from the lesion. If they are, the field strength of the electrical field at the lesion for a given magnitude of current will be reduced. Therefore, the magnitude of the current would have to be increased to yield the same electrical field strength at the lesion.

For optimal results in a human patient, uniform electrical field of the desired strength is imposed over about 10 cm to 20 cm of damaged spinal cord surrounding the lesion for a beneficial clinical outcome. Ideally, this uniform field is imposed across the entire cross section of the spinal cord over this longitudinal extent, because of the general segregation of descending (motor) tracts to the ventral (anterior) cord, and the segregation of important (largely sensory) tracts to the posterior (dorsal) spinal cord. Circuit 700 is configured to facilitate provision of such a uniform field. This uniform electrical field of the desired strength may be generated by placing two pairs of electrodes, for example electrodes 340, 346, 342, and 348, on either side (two tethered to the right and left lateral facets) and a third pair, for example electrodes 344 and 350, sutured to the paravertebral muscle and fascia of the dorsal (posterior) facet rostrally and caudally of the spinal cord lesion. Additionally, a fourth pair of electrodes, for example 384 and 386, are sutured to paravertebral musculature at the extreme mediolateral/ventral (anterior) vertebral column. The placement of this fourth pair of electrodes 384 and 386 should alleviate the reduction of the voltage gradient imposed over motor columns in the anterior (ventral) spinal cord.

Once inside a patient, it is difficult to verify the operation of a device comprising circuit 300, 600, 700, 800. Visible verification is impossible while the device is within a patient. Operation of the device within the patient could be determined by attaching an electrocardiogram (EKG) system to the patient and waiting to observe a small transient on the EKG record associated with the reversal of the electrical field imposed over the spinal cord, but this is a time consuming procedure.

Optional beacon circuit 320 can be used with circuit 300, 600, 700 or 800 to enable rapid verification of device operation. Beacon circuit 320 can be any circuit that enables visible and/or audible verification of device operation. Beacon circuit 320 also can transmit data regarding device operation, such as, for example, using RF telemetry.

In an embodiment, a small LED “beacon” is inserted into circuit 300, 600, 700, and 800. A periodic visible burst of light such as, for example, every 7 seconds, reveals nominal unit operation prior to implantation. After implantation this burst of light may in certain circumstances be visible transdermally.

In an embodiment, a low-frequency oscillator connected to a small-coil antennae within the device unit enables verification of operation following device implantation. A pulsed signal is transmitted by the oscillator/antennae. A small acoustic amplifier placed near the implantation site on the patient amplifies this signal and audiblizes it as a “chirp”.

FIG. 4 shows a schematic of an embodiment of beacon circuit 320 of the disclosed device. Beacon circuit 320 comprises electronic components electrically interconnected as shown in FIG. 4. Conventional symbols are used to denote the components. Nodes 325 and 327 are shown in FIG. 4 to define the connection points between circuit 300, 700 and 800 and beacon circuit 320. The beacon circuit 320 may also be connected to circuit 600 in a similar manner. As shown, for example in FIG. 4 the illustrated light emitting embodiment of beacon circuit 320 includes a light emitting diode 402, a transistor 404, resistors 406, 408, 410 and 412, capacitors 414, 416, 418, 420, and 422 and inductor 424. In the illustrated embodiment, various commercially available electronic components may be utilized to implement the disclosed beacon circuit 320. For example, in particular, transistor 404 may be an MMBT 3904 NPN General Purpose Amplifier available from Fairchild Semiconductor Corporation, South Portland, Me.

As shown, for example, in FIG. 4, the collector of the transistor 404 is coupled to a node to which one electrode of the inductor 424, and the positive electrode of the capacitor 420 are connected. The other electrode of the inductor 424 is coupled to node 325 which is coupled to the positive voltage terminal 310 (FIG. 3). The negative electrode of the capacitor 420 is coupled to a node 426 coupled to the emitter of transistor 404, one electrode of resistor 412 and the negative electrode of capacitor 422. The positive electrode of capacitor 422 is coupled to node 325. The illustrated arrangement of capacitor 420, capacitor 422, and inductor 424 form an oscillator tank which in conjunction with transistor 404 determines the oscillator frequency of the oscillator. In the illustrated embodiment, inductor exhibits a 220 microHenry inductance, and capacitors 420 and 422 each exhibit a 0.047 microfarad capacitance inducing the oscillator to oscillate at approximately 70 kHz which produces an electromagnetic pulse that is detectable by a pick-up coil such as that shown in FIG. 5 so that proper operation of the circuit 300, 600, 700 or 800 to which the beacon circuit 400 is connected can be verified, either before or after implantation of the device.

The other electrode of resistor 412 is coupled to a node 428 to which one electrode of resistor 410, the negative electrode of capacitor 418 and the positive electrode of capacitor 416 is coupled. the other electrode of resistor is coupled to the base of the transistor 404 and to one electrode of resistor 408. The other electrode of resistor 408 is coupled to node 325. Resistors 408 and 410 are coupled and configured to define a voltage divider dividing the voltage between node 325 and node 428. The positive electrode of capacitor 418 is coupled to node 325. The negative electrode of capacitor 416 is coupled to node 327 which is coupled to the Q7 pin of the timer 382 (FIG. 3). The cathode of the diode 402 and one electrode of resistor 406 are coupled to node 325. The other electrode of resistor 406 and the anode of the light emitting diode are coupled to the positive electrode of the capacitor 414. The negative electrode of the capacitor 414 is coupled to node 327.

When configured as shown in FIG. 4, the beacon circuit 320 is configured to cause the light emitting diode 402 to flash on for a period at a frequency determined by the output of the Q7 pin of timer 382 (or counter 602 when coupled to circuit 600). Likewise, the beacon circuit 320 causes the oscillator to oscillate for this same period The duty cycle of the light emitting diode 402, the brightness of the emitted light and the frequency of the oscillator are established by, among other things, the values of the resistors 406, 408, 410, 412, capacitors 414, 416, 418, 420, 422 and the inductor 424, the transistor 404 and light emitting diode 402 selected and the output of the Q7 pin of the timer 382.

FIG. 5 shows a schematic of a receiver circuit 500 according to one embodiment of the disclosed device for use in conjunction with the beacon circuit 320 of FIG. 4. Receiver circuit 500 comprises electronic components electrically interconnected as shown in FIG. 5. Conventional symbols are used to denote the components. Receiver circuit 500 as shown in FIG. 5 comprises function generator 502, modulator/demodulator 504, and amplifier 506, a pickup coil 508, a transistors 510 and 512, speaker 514, batteries 516 and 518 and various resistors, potentiometers, and capacitors configured as shown. The values of the various components including the values of the resistors and capacitors and the settings of the potentiometers are selected to power and tune the receiver circuit according to the desired sensitivity frequency of the receiver circuit 500.

Various commercially available electronic components may be utilized to implement receiver circuit 500. In one embodiment of receiver circuit 500, function generator 502 is an XR2206 Monolithic Function Generator available from Exar Corporation, Fremont Calif.

In one embodiment of receiver circuit 500, modulator/demodulator 504 is an MC1496 Balanced Modulators/Demodulators available from ON Semiconductor, Denver, Colo. Other modulator/demodulators may be use in circuit 500 within the scope of the disclosure. The modulator/demodulator 504 is designed for use where the output voltage is a product of an input voltage (signal) and a switching function (carrier) generated by the function generator 502.

In one embodiment of receiver circuit 500, amplifier 506 is an LM386 Low Voltage Audio Power Amplifier available from National Semiconductor Corporation, Santa Clara, Calif.

In one embodiment of receiver circuit 500, pickup coil 508 is formed by coiling 200 turns of #34 wire into a 2.5 inch diameter coil on a four foot coaxial cable.

In one embodiment of receiver circuit 500, transistors are 2N3904 NPN General Purpose Amplifier transistors, from Fairchild Semiconductor Corporation, South Portland, Me.

In one embodiment of receiver circuit 500, batteries 516 and 518 are 9 volt batteries.

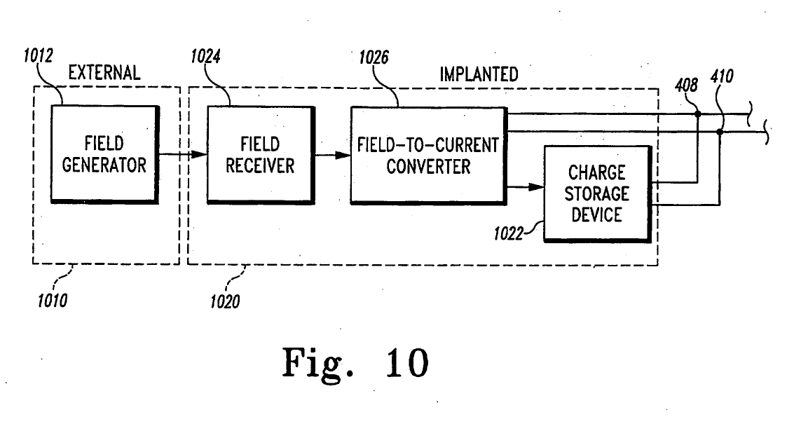

FIG. 10 shows a block diagram of a schematic of a second embodiment of the circuit 300. This second embodiment comprises an external portion 1010 and an internal portion 1020. The external portion comprises a field generator 1012 that is configured to generate an electric, magnetic, or electromagnetic field. The 1020 comprises a field receiver 1024, a field-to-current converter 1026 and a charge storage device 1022.

In operation, external portion 1010 operates as an electric or magnetic field generator 1012. The field may also be alternating current or radio frequency, in which case it will be coupled wirelessly, by means of inductive or capacitive coupling to the field receiver 1024. The field receiver 1024 may be two conductive leads that receive charge from the field generator 1012. Alternatively, field receiver 1024 may be a conductive coil onto which a magnetic field will be coupled from the field generator 1012. Alternatively, field receiver 1024 may be a capacitive plate onto which an electric field will be coupled from the field generator 1012.

The field-to-current converter 1026, may operate to transform magnetically or electrically coupled fields to direct current fields through charge-rectifying and/or signal conditioning. The field-to-current converter 1026 may also regulate coupled power delivery for appropriate charging of the charge storage device 1022. Simultaneously, during charging, the field-to-current converter 1026 can also supply power to the nodes 808 and 810 of the circuit 300, in addition to the charge-storage device 1022.

The charge storage device 1022 may be a rechargeable battery, such as the rechargeable battery 802, or a capacitor. The charge storage device 1022 may store power received from the field-to-current converter 1022 up to its maximum capacity, which is monitored by the field-to-current converter 1022 to avoid over-charging of the charge storage device 1022. Upon reaching maximum capacity, the charge storage device 1022 may contain enough power to power the circuit 300 via the nodes 808 and 810 for the appropriate length of time, and charging may cease.

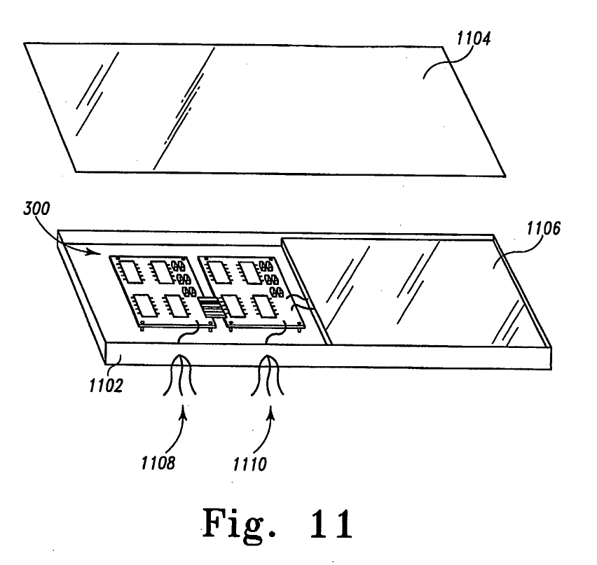

FIG. 11 show an embodiment of a case 1100 for use with the circuits 300, 600, 700 and 800. The embodiment of case 1100 shown in FIG. 11 is a hexahedron, but other geometries are within the scope of the disclosure. The case 1100 comprises a bottom portion 1102 and a top portion 1104. The portions 1102 and 1104 of the case 1100 may be manufactured of one or more suitable materials, such as stainless steel, titanium, Nitinol, platinum-iridium, borosilicate, quartz, ceramic or silicone. The bottom portion 1102 and the top portion 1104 of case 1100 may be laser welded together to form a seal, or may be coupled together with an adhesive, such as an epoxy or glue.

The circuit 300, 600, 700, 800 which may comprise one or more circuit boards, may be coupled to the case 1100. The circuit 300 is shown coupled to the bottom portion 1102 of the case 1100 in FIG. 11, but the circuit 300, 600, 700, 800 may be coupled to any part of the case, or may even be held in place by a total or partial encasement in a hardening liquid or gel, such as epoxy or plastic. A charge storage device 1106 may also be coupled to the case 1100. The charge storage device 1106 may be held in place by an adhesive or mechanical fastener, or may even be manufactured as an integral component of the case 1100 or circuit 300, 600, 700, 800.

One or more orifices 1108 and 1110 in on or more walls of the case 1100 may allow a first plurality of electrodes, such as electrodes 340, 342 and 344, and a second plurality of electrodes, such as electrodes 346, 348 and 350, to extend from the interior to the exterior of the case 1100. The one or more orifices 1108 and 1110 are shown illustratively in FIG. 11 in a side wall of the bottom portion 1102 of the case 1100, but one or more orifices 1108, 1110 may be located anywhere within the case 1100.

The case 1100 may enable long term (greater than one year) implantation of the circuit 300, 600, 700, 800 within patients. In some embodiments, case 1100 comprises of lithium, ceramic-based materials and/or medical grade alloys of stainless steel. Titanium is used in one preferred embodiment, and illustrative case 1100 may comprise pure medical grade titanium. The case 1100 may be one of the variety of sizes and shapes of cases commercially provided by Medtronic, Inc., of Minneapolis, Minn. or Boston Scientific Corp. of Boston, Mass. Alternatively, the case 1100 may comprise a titanium tube having an outer diameter and an inner diameter, as available in various sizes from LN Industries S.A., Grandeson, Switzerland. The individual portions 1102 and 1104 of the case 1100 may be laser machined and welded together to form a hermetically sealed barrier to fluids after the circuit 300, 600, 700, 800 is placed inside during assembly. For example, Laserage Technology of Wakegan, Ill. provides laser welding of titanium cases.

The overall size of the case 1100 may be on the order of about 4 cm×3 cm×2 cm with a wall thickness of about 0.6 mm to about 7 cm×6 cm×3 cm with a wall thickness of about 0.7 mm. The orifices 1108 and 1110 may be pre-machined holes, and may be sealed by conventional glass and/or titanium annealing, elastomer or polycarbonate seals to act as fluid barriers after assembly. In another embodiment the electrodes may be soldered to externalized micro-dot gold or titanium connectors, and the joints protected with medical grade elastomer or sealant. Welding of the case 1100 may be accomplished with YAG lasers. Serial numbers and other identifiers can also be etched by laser or other engraving techniques onto the surface of the case 1100.

While this invention has been described as having a preferred design, the present invention can be further modified within the scope and spirit of this disclosure. This application is therefore intended to cover any variations, uses, or adaptations of the invention using its general principles. For example, the methods disclosed herein and in the appended claims represent one possible sequence of performing the steps thereof. A practitioner of the present invention may determine in a particular implementation of the present invention that multiple steps of one or more of the disclosed methods may be combinable, or that a different sequence of steps may be employed to accomplish the same results. Each such implementation falls within the scope of the present invention as disclosed herein and in the appended claims. Furthermore, this application is intended to cover such departures from the present disclosure as come within known or customary practice in the art to which this invention pertains and which fall within the limits of the appended claims.

1. An apparatus for stimulating axon growth of the nerve cells in the spinal cord of mammals, the apparatus comprising:
a constant current DC stimulus generator the generator having first and second groups of oppositely polarized output terminals wherein one of the first and second groups of output terminals comprises a cathode and the other of the first and second groups of output terminals comprises an anode of the generator;
first and second groups of electrodes electrically coupled respectively to the first and second groups of output terminals;
a beacon signal generator electrically coupled to the DC stimulus generator; and
a polarity reversing circuit electrically coupled to the constant current DC stimulus generator and configured to reverse the polarity of the DC stimulus each time a predetermined period of time elapses, wherein each time the polarity of the DC stimulus is reversed the output terminal which comprised the cathode before the polarity reversal comprises the anode after the reversal and the output terminal which comprised the anode before the polarity reversal comprises the cathode after the polarity reversal.

2. The device of claim 1 and further comprising a current chopping circuit electrically coupled to the constant current DC stimulus generator and configured to turn on and off the current produced by the DC stimulus generator so that the current produced exhibits a duty cycle.

3. The device of claim 1 wherein the predetermined period of time is less than a period of time after which an anode-facing axon begins to experience die back.

4. The device of claim 3 wherein the predetermined period of time is in the range between about ten minutes and about twenty minutes.

5. The device of claim 1 and further comprising a charge storage device electrically coupled to the constant current DC stimulus generator.

6. The device of claim 5 wherein the charge storage device is rechargeable.

7. The device of claim 5 and further comprising a case having a top portion and a bottom portion, wherein the constant current DC stimulus generator and the charge storage device are positioned between the top portion and bottom portion.

8. The device of claim 1 and further comprising a failsafe circuit electrically coupled to the polarity reversing circuit and the DC stimulus generator and configured to interrupt the output of the DC stimulus generator upon sensing a failure of the polarity reversing circuit.

9. The device of claim 1 wherein the polarity reversing circuit utilizes a clock pulse to monitor the passage of the predetermined period of time and further comprising a failsafe circuit electrically coupled to the DC stimulus generator and the polarity reversing circuit and configured to interrupt the output of the DC stimulus generator upon sensing an interruption of the clock pulse.

10. An apparatus for stimulating axon growth of the nerve cells in the spinal cord of mammals, the apparatus comprising:
a stimulus generator capable of generating a chopped DC current, the generator having first and second oppositely polarized output terminals wherein one of the first and second output terminals comprises a cathode and the other one of the first or second output terminals comprises an anode of the generator;
first and second electrodes electrically coupled respectively to the first and second output terminals; and
a polarity reversing circuit electrically coupled to the stimulus generator configured to reverse the polarity of the stimulus each time a predetermined period of time elapses, wherein each time the polarity of the stimulus is reversed the output terminal which comprised the cathode before the polarity reversal comprises the anode after the reversal and the output terminal which comprised the anode before the polarity reversal comprises the cathode after the polarity reversal.

11. The device of claim 10 wherein the stimulus generator comprises a constant current DC stimulus generator and a current chopping circuit electrically coupled to the constant current DC stimulus generator and configured to turn off the current for an off duty cycle.

12. The device of claim 10 and further comprising a rechargeable charge storage device electrically coupled to the stimulus generator.

13. The device of claim 12 and further comprising a case having a top portion and a bottom portion, wherein the stimulus generator and the charge storage device are positioned between the top portion and bottom portion.

14. A method for stimulating axon growth of the nerve cells in the spinal cord of mammals, the method comprising:
providing a device for stimulating axon growth of the nerve cells in the spinal cord of mammals, the device comprising:
a constant current DC stimulus generator, the generator having first and second groups of oppositely polarized output terminals wherein one of the first and second groups of output terminals comprises a cathode and the other of the first and second groups of output terminals comprises an anode of the generator;
first and second groups of electrodes electrically coupled respectively to the first and second groups of output terminals;
a beacon signal generator electrically coupled to the DC stimulus generator; and
a polarity reversing circuit electrically coupled to the constant current DC stimulus generator and configured to reverse the polarity of the DC stimulus each time a predetermined period of time elapses, wherein each time the polarity of the DC stimulus is reversed the output terminal which comprised the cathode before the polarity reversal comprises the anode after the reversal and the output terminal which comprised the anode before the polarity reversal comprises the cathode after the polarity reversal; and
implanting the device in a mammal.

15. The method of claim 14 and further comprising generating an electrical field with the provided device along the spinal cord of the mammal in which the device is implanted.

16. The method of claim 15 and further comprising reversing the polarity of the generated electrical field utilizing the polarity reversing circuit of the provided device.

17. The method of claim 16 repeatedly reversing the polarity of the generated electrical field each time a predetermined period of time passes since the last reversal of polarity of the generated electrical field.

18. The method of claim 17 and further comprising setting the predetermined period of time to be less than the time required for an anode-facing axon to begin to die back under the influence of the generated electrical field.

19. The method of claim 17 and further comprising sensing a beacon signal generated by the beacon signal generator after the implanting step.

20. A method for stimulating axon growth of the nerve cells in the spinal cord of mammals, the method comprising:
providing a device for stimulating axon growth of the nerve cells in the spinal cord of mammals, the device comprising:
a stimulus generator capable of generating a chopped DC current, the generator having first and second oppositely polarized output terminals wherein one of the first or second output terminals comprises a cathode and the other one of the first or second output terminals comprises an anode of the generator;
first and second groups of electrodes electrically coupled respectively to the first and second output terminals; and
a polarity reversing circuit electrically coupled to the stimulus generator configured to reverse the polarity of the stimulus each time a predetermined period of time elapses, wherein each time the polarity of the stimulus is reversed the output terminal which comprised the cathode before the polarity reversal comprises the anode after the reversal and the output terminal which comprised the anode before the polarity reversal comprises the cathode after the polarity reversal; and implanting the device in a mammal.

21. An apparatus for stimulating axon growth of the nerve cells in the spinal cord of mammals, the apparatus comprising:
a constant current DC stimulus generator, the generator having first and second groups of oppositely polarized output terminals wherein one of the first or second groups of output terminal comprises a cathode and the other one of the first or second groups of output terminals comprises an anode of the generator;
first and second groups of electrodes electrically coupled respectively to the first and second groups of output terminals;
a rechargeable charge storage device electrically coupled to the constant current DC stimulus generator; and
a polarity reversing circuit electrically coupled to the constant current DC stimulus generator configured to reverse the polarity of the DC stimulus each time a predetermined period of time elapses, wherein each time the polarity of the DC stimulus is reversed the output terminal which comprised the cathode before the polarity reversal comprises the anode after the reversal and the output terminal which comprised the anode before the polarity reversal comprises the cathode after the polarity reversal.

22. The device of claim 21 and further comprising a current chopping circuit electrically coupled to the constant current DC stimulus generator and configured to turn off the current for an off duty cycle and turn on the current for an on duty cycle.

23. The device of claim 21 further comprising a case having a top portion and a bottom portion, wherein the constant current DC stimulus generator and the charge storage device are positioned between the top portion and bottom portion.

24. An apparatus for stimulating axon growth of the nerve cells in the spinal cord of mammals, the apparatus comprising:
a constant current DC stimulus generator, the generator having first and second groups of oppositely polarized output terminals wherein one of the first or second groups of output terminal comprises a cathode and the other one of the first or second groups of output terminals comprises an anode of the generator;
first and second groups of electrodes electrically coupled respectively to the first and second groups of output terminals;
a charge storage device electrically coupled to the constant current DC stimulus generator;
a case having a top portion and a bottom portion, wherein the constant current DC stimulus generator and the charge storage device are positioned between the top portion and bottom portion; and
a polarity reversing circuit electrically coupled to the constant current DC stimulus generator configured to reverse the polarity of the DC stimulus each time a predetermined period of time elapses, wherein each time the polarity of the DC stimulus is reversed the output terminal which comprised the cathode before the polarity reversal comprises the anode after the reversal and the output terminal which comprised the anode before the polarity reversal comprises the cathode after the polarity reversal.

25. The device of claim 24 and further comprising a current chopping circuit electrically coupled to the constant current DC stimulus generator and configured to turn on and off the current produced by the DC stimulus generator so that the current produced exhibits a duty cycle.

26. The device of claim 24 further comprising a rechargeable charge storage device electrically coupled to the constant current DC stimulus generator.

27. The device of claim 24 wherein each of said first and second groups of electrodes comprises a first electrode corresponding to a first electrode of the other of the first and second groups, a second electrode corresponding to a second electrode of the other of the first and second groups, a third electrode corresponding to a third electrode of the other of the first and second groups, and a fourth electrode corresponding to a fourth electrode of the other of the first and second groups.

28. The device of claim 27 and further comprising a failsafe circuit electrically coupled to the polarity reversing circuit and the DC stimulus generator and configured to interrupt the output of the DC stimulus generator upon sensing a failure of the polarity reversing circuit.

29. An apparatus implanted in a mammalian body having a spine and a lesion in the spinal cord for stimulating axon growth of the nerve cells in the spinal cord of mammals, the apparatus comprising:
a constant current DC stimulus generator, the generator having first and second groups of oppositely polarized output terminals wherein one of the first and second groups of output terminals comprises a cathode and the other of the first and second groups of output terminals comprises an anode of the generator;
first and second groups of electrodes electrically coupled respectively to the first and second groups of output terminals, each of said first and second groups of electrodes having a first electrode corresponding to a first electrode of the other of the first and second groups, a second electrode corresponding to a second electrode of the other of the first and second groups, a third electrode corresponding to a third electrode of the other of the first and second groups, and a fourth electrode corresponding to a fourth electrode of the other of the first and second groups;
a polarity reversing circuit electrically coupled to the constant current DC stimulus generator and configured to reverse the polarity of the DC stimulus each time a predetermined period of time elapses, wherein each time the polarity of the DC stimulus is reversed the output terminal which comprised the cathode before the polarity reversal comprises the anode after the reversal and the output terminal which comprised the anode before the polarity reversal comprises the cathode after the polarity reversal,
wherein the first electrodes of the first and second group of electrodes are positioned on the right lateral facet of the spine of the mammalian body with the electrode of the first group of electrodes positioned rostrally from the lesion and the electrode of the second group of electrodes positioned caudally from the lesion, the second electrodes of the first and second group of electrodes are positioned on the left lateral facet of the spine of the mammalian body with the electrode of the first group of electrodes positioned rostrally from the lesion and the electrode of the second group of electrodes positioned caudally from the lesion, the third electrodes of the first and second group of electrodes are positioned on the paravertebral muscle and fascia of the dorsal (posterior) facet of the mammalian body with the electrode of the first group of electrodes positioned rostrally from the lesion and the electrode of the second group of electrodes positioned caudally from the lesion, and the fourth electrodes of the first and second group of electrodes are positioned adjacent to paravertebral musculature at the extreme mediolateral/ventral (anterior) vertebral column of the mammalian body with the electrode of the first group of electrodes positioned rostrally from the lesion and the electrode of the second group of electrodes positioned caudally from the lesion.

30. The device of claim 29 wherein the constant current DC stimulus generator and the first and second groups of electrodes cooperate to generate a substantially uniform electrical field over about 10 cm to 20 cm of damaged spinal cord surrounding the lesion.

31. The device of claim 30 wherein the substantially uniform field is imposed across substantially the entire cross section of the spinal cord.

32. The device of claim 31 and further comprising a current chopping circuit electrically coupled to the constant current DC stimulus generator and configured to turn on and off the current produced by the DC stimulus generator so that the current produced exhibits a duty cycle.
